(19) United States
(12) Patent Application Publication
(10) Pub. No.: US 2020/0338309 A1
(43) Pub. Date: Oct. 29, 2020

(54) System and Method for Automated Cannulation

(71) Applicant: FRESENIUS MEDICAL CARE DEUTSCHLAND GMBH, Bad Homburg (DE)

(72) Inventors: Pascal KOPPERSCHMIDT, Dittelbrunn (DE); Pia DANIEL, Bodman (DE); Reiner SPICKERMANN, Wasserlosen-Burghausen (DE); Otto ARKOSSY, Budapest (HU); Cacilia SCHOLZ, Schwalbach (DE); Kai-Uwe RITTER, Rednitz-Hembach (DE); Elke SCHULTE, Schweinfurt (DE); Christopher HAUKE, Mainz-Kostheim (DE)

(73) Assignee: FRESENIUS MEDICAL CARE DEUTSCHLAND GMBH, Bad Homburg (DE)

(21) Appl. No.: 16/479,608

(22) PCT Filed: Jan. 29, 2018
(86) PCT No.: PCT/EP2018/052148

§ 371 Date: Jul. 22, 2019

(30) Foreign Application Priority Data
Jan. 30, 2017 (DE) 10 2017 201 437.7

(51) Int. Cl. A61M 25/01; A61M 1/36; A61B 8/08; A61B 6/00; A61B 5/00; A61B 5/02

(52) U.S. Cl. CPC A61M 25/0116; A61M 1/3661; A61B 8/0891; A61B 2034/301; A61B 5/0082; A61B 5/489; A61B 5/02007; A61B 6/504

(57) ABSTRACT

The invention relates to a system (100) for data-dependent automated cannulation of patient blood vessels, in particular for hemodialysis, comprising: at least one cannulation robot (1) configured for automated cannulating of patient blood vessels, a control system (50, 51) comprising at least one data processing device and which is configured to implement a control procedure which controls the at least one cannulation robot subject to program parameters, at least one user interface device (80), enabling user input by means of which a patient is registered in the control system (50, 51), whereby in consequence of this control system registration procedure, an individually assigned patient identifier, which is referred to as the registered patient identifier, is used for the registered patient, and wherein the control system is configured to define the program parameters as a function of the registered patient identifier and control the at least one cannulation robot (1) as a function of the registered patient identifier. The invention furthermore relates to a corresponding method.

The invention relates to a system for the automated cannulation of patient blood vessels and a corresponding method, in particular for hemodialysis.

The venipuncture of blood vessels, also known as cannulation, is a routine procedural step in the medical treatment of many patients in which a fluid connection, in particular a cannula, is established between a patient's blood circulation and an external fluid system. Cannulation is performed in the medical practice of physicians or trained personnel. The quality of the vascular access created by the cannulation thereby depends on a plurality of parameters which are in particular affected by the individual and temporally varying abilities of the medical personnel and the physical characteristics of the patients to be treated as well as the diversity of the technical instruments used in cannulation. In order to standardize cannulation, cannulation robots have been developed which autonomously prepare a patient cannulation procedure and autonomously perform the cannulation using suitable sensor technology and motor function. Such cannulation robots and the technical resources thereby used are known from e.g. US 2015/0065916 A1, WO 2015/052719 A1 and WO 2010/029521 A2.

Chronically ill patients need regularly repeated vasculature cannulation in order to ensure the necessary treatment. One such chronic illness is kidney failure which leads, among other things, to the loss of the blood's natural purifying function. Technical solutions can be substituted in its place. Hemodialysis devices are extracorporeal filtering units serving as artificial kidneys into which the blood of the patent is conducted in order to be cleansed and treated before being returned to the patient's blood circulation. Blood is normally withdrawn and returned via an artificial subcutaneous connection surgically created between a vein and an artery in an arm or a leg of the patient. This connection can be composed of a section of the patient's own vasculature prepared for same or can consist of an artificial material and is referred to as a fistula or arteriovenous fistula respectively (AV fistula, AVF).

The most commonly used permanent vascular access in chronic hemodialysis patients is a native arteriovenous fistula. After the native arteriovenous fistula is placed, it become stronger due to the increased blood flow, whereby repeated cannulation for the dialysis treatment becomes simpler.

Hemodialysis must be performed regularly, typically a few days apart. This leads to high mechanical stress on the blood vessel or arteriovenous fistula respectively. Different techniques are known to create access to a blood vessel or arteriovenous fistula respectively, these aiming to be as gentle as possible on the vessel over the course of the repeated cannulation. In rope ladder cannulation, a new cannulation site located at a distance from the previous site, e.g. about 2 cm, is sought for each treatment. In this method, the series of punctures are usually started at the lower end of the vessel and then continue upward until reaching the upper end and the process then started again from below. The practitioner must thereby precisely follow the positioning pattern so as to allow the venipunctured vessel sites to heal. In contrast, in the buttonhole technique, a needle is always inserted into the exact same spot at the exact same angle. Over time, a scar tunnel thus develops which continually displaces the thrombus forming in cannulation and thus becomes more resilient. It has been found that buttonhole technique results can be improved if the cannulation is always performed by the same treatment personnel.

Due to the frequency of cannulation with hemodialysis patients, the arteriovenous fistula is subject in general to high stress, independent of the venipuncture technique, same which can lead to changes in the surface of the skin and the condition of the arteriovenous fistula and how they progress.

The analysis of observations of hemodialysis patients which underlies the present invention revealed that the condition and progression of the arteriovenous fistula over the course of treatment can widely vary from patient to patient. This makes cannulation in hemodialysis patients a relatively complex procedure which poses challenges to experienced treatment personnel. Even the configuration of cannulation robots in such situations requires a great deal of effort.

The present invention is based on the task of specifying an efficient technical solution for cannulation under variable operating conditions, as is in particular found with hemodialysis.

Serving in solving this task is the system for automated cannulation according to claim 1 and the method for automated cannulation according to claim 15. Preferential embodiments of the invention constitute the subject matter of the subclaims and the present description of the invention.

The inventive system for data-dependent automated cannulation of patient blood vessels, in particular for hemodialysis, comprises: at least one cannulation robot configured for automated cannulating of patient blood vessels, at least one control system comprising at least one data processing device and which is configured to implement a control procedure which controls the at least one cannulation robot subject to program parameters, at least one interface device enabling user input into the control system by means of which a user is registered in the control system, whereby in consequence of this control system registration procedure, a patient identifier which is individually assigned to the registered patient and which is referred to as the registered patient identifier, is used, and wherein the at least one control system, in particular the at least one control procedure, is configured to define the program parameters as a function of the registered patient identifier and control the at least one cannulation robot as a function of the registered patient identifier.

The inventive system is configured to individually adapt the automated cannulation to the specific baseline conditions of a specific patient. A majority of this adaptation can ensue as preparation prior to the automated cannulation procedure and therefore does not need to take place during the cannulation. This is made possible by the fact of the patient having previously registered in the system via a user interface. Such a system is particularly efficient to use and offers the advantage of expanded application and design possibilities. In particular, such a system provides the advantage of being able to identify extensive patient data and provide it for data storage for the system to use in subsequent automated cannulation treatments in order to further improve the reliability.

In particular as a system for the cannulation of blood vessels, in particular native arteriovenous fistulas, in hemodialysis patients, the system provides the advantage of the precision of cannulation robots in combination with their patient-specific and data-based control resulting in particularly reliable venipuncture results while at the same time protecting the vessels over the course of repeated hemodialysis treatments.

The process steps of the automated cannulation begin as defined by the present invention and described in the following after the part of the patient's body to be venipunctured has been inserted into a treatment chamber of the cannulation robot, e.g. by the patient himself or by medical treatment personnel.

A cannulation robot is an apparatus which automatically; i.e. intermittently or continuously, performs at least one cannulation process step in a patient blood vessel, or several or all intended process steps, without the intervention of a human operator, e.g. medical personnel. This thereby ensues in particular by the program parameters of the automated cannulation being accordingly selected by the system and/or by the user. One process step in the cannulation is in particular technically implemented by a component of the cannulation robot, e.g. a tool device, specifically configured for said process step and is selected from the group comprising the possible process steps P1, P2, P3 . . . , without this numbering defining a sequential ordering:
P1: Using an equipment kit to perform the cannulation which is selected prior to commencing the automated cannulation based on the registered patient identifier; this selection can have been made previously by means of an optional pick-and-place system of the system for selecting an equipment kit and/or equipping an equipment holder, in particular an equipment box; the equipment kit can have been provided beforehand as a function of the registered patient identifier by an optional sorting apparatus of the system selecting the equipment contained in the equipment kit from an optional storage apparatus of the system for storing equipment; the equipment kit can contain one or more medical equipment items, in particular gauze, swabs, adhesive tape; the equipment of this equipment kit can be gathered as a function of the registered patient identifier and/or as a function of patient-specific treatment data derived from the registered patient identifier; the use of this equipment kit by the cannulation robot is a process step of the automated cannulation and can provide for the equipment of the equipment kit to be automatically extracted from predetermined positions of an equipment holder/box, in particular by the appropriate program parameters being selected as a function of the registered patient identifier and suitable for extraction; an optional pick-and-place device of the cannulation robot being in particular used to that end which is configured to extract the equipment out of the equipment holder and/or configured to equip one or more optional tool devices of the cannulation robot;
P2: Spatially fixating a part of the patient's body containing the blood vessel, in particular an arteriovenous fistula; the program parameters of the automated cannulation can be selected here as a function of the registered patient identifier, thus individual to each patient, these program parameters setting beforehand the position or the spacing of one or more optional fixation devices of the cannulation robot based on a previously determined location or on pre-determined spacings on the patient's body part so as to achieve suitable fixation; the fixation taking place in the treatment chamber of the cannulation robot in which the patient's body part rests for the at least one ensuing cannulation;
P3: Using stored—in particular in a patient database—patient data in order to determine information on past cannulation process steps in the patient's vasculature (historical data), and preferably define the cannulation to occur, in particular the program parameters thereby used, based on this historical data; such historical data containing in particular the location of one or more of the patient's blood vessels previously measured by an optional measuring device of the cannulation robot for measuring the location and/or dimensions of at least one blood vessel under the patient's skin (vascular structure measuring device) and providing same in particular as patient data; such historical data containing in particular information on the location and condition of further venipuncture sites on the patient's body which is in particular provided as patient data; the vascular structure measuring device being able to be designed to detect the location and/or dimensions of at least one blood vessel under the patient's skin by means of ultrasound or by means of optical radiation;
P4: Identifying the blood vessel under the patient's skin suitable for the blood withdrawal, in particular selecting a suitable insertion site on the skin for the cannulation of said blood vessel; the program parameters of the automated cannulation can hereby be selected as a function of the registered patient identifier, thus individual to the specific patient, by the cannulation planned for the registered patient being selected on the basis of at least one patient-specific treatment parameter; for example with a patient planned for hemodialysis; a treatment parameter can encode the patient's necessity for hemodialysis; the cannulation of an arteriovenous blood vessel can be planned by evaluating the treatment parameter; same being identified; the identification can for example ensue in the control system by a program-controlled analysis of an image obtained by a vascular structure measuring device;
P5: Disinfecting the skin of the patient's body part containing the blood vessel; the program parameters of the automated cannulation can hereby be selected as a function of the registered patient identifier, thus individual to the patient, by a disinfecting process being specifically selected for the patient's type of skin or skin morphology which is for example characterized by the length of the treatment or the amount and nature of the disinfecting process employed; treatment data specific to the patient can also be considered; a disinfecting device which is optional with the cannulation robot or separate therefrom and equipped to perform the cited function can be used for the cited disinfection; the type of skin or skin morphology of the patient being preferably known in particular as patient data in the patient database;
P6: Physically treating the patient's body part containing the blood vessel in preparation for the cannulation, in particular stemming the blood flow of the body part, applying pressure to the body part, controlling the temperature of the body part, positioning the immobilized body part; the program parameters of the automated cannulation can hereby be selected as a function of the registered patient identifier, thus individual to the specific patient, by drawing on preparation data specific to the planned patient treatment, e.g. hemodialysis, or which can be taken from the patient database as known preparation data; this preparing for the cannulation of the body part being in particular performed by an optionally provided prepping device of the cannulation robot correspondingly configured for this purpose;
P7: Particularly preferential: Puncturing the blood vessel, in particular an arteriovenous fistula; preferably a first venipuncture and cannulation occurring automatically for withdrawing blood from the blood vessel and a second venipuncture and cannulation occurring automatically for the return of the blood, in particular in the case of hemodialysis; the program parameters of the automated cannulation can hereby be selected as a function of the registered patient identifier, thus individual to the specific patient, by the program parameters defining a patient-dependent motion control for a robotic tool arm optionally provided in the cannulation robot, by means of which medical equipment such as for instance an injection needle can for example be grasped by the tool arm and positioned on the body part, with the injection needle having been previously selected and prepared specific to the patient; two cannulation robots can be set up for venipuncturing blood vessels at different parts of the body by, for example, a first cannulation robot being configured for cannulation on an arm and a second cannulation robot being configured for cannulation on a leg; the selection of the appropriate cannulation robot can ensue in patient-specific and/or treatment-specific manner;
P8: Withdrawing blood from the cannulated blood vessel and transporting the blood in at least one blood transport device or in at least one sample container; the program parameters of the automated cannulation can hereby be selected as a function of the registered patient identifier, thus individual to the specific patient, by a suitable blood transport device or suitable sample container being preselected as a function of patient-specific treatment data and then utilized in suitable manner by the cannulation robot; the cannulation robot and the control system can be configured thereto by an appropriate selection of the program parameters to provide at least one sample container based on treatment data for the subsequent, preferably automatic and system-controlled, treatment, in particular diagnostics.

The program parameters used for the automated cannulation based on the registered patient identifier are selected via a selection process. The selection process is implemented by the control system, in particular by a control device of a cannulation robot, and can be part of the control procedure. The selection process can be implemented as a program code executable by a data processor of the control system.

The selection process can be configured to access a data matrix in which the necessary program parameters are linked as a function of treatment data and thereby identifiable. This data matrix can be stored in a database of the system. In addition, the selection process can be configured to determine at least one program parameter by calculation algorithm. A calculation algorithm can for example be configured to calculate the required volume of blood to be obtained by cannulation based on the number and type of planned process steps, or blood test steps respectively, to be preferably automatically performed by the system by way of cannulation following provision of the blood sample.

The system, preferably the control system, in particular a control apparatus of the control system and/or a control device of an apparatus of the system and/or the control procedure, is preferably configured to perform at least one preparatory procedural step after acquiring the patient identifier and prior to the start of the automated cannulation in preparation of the automatic withdrawal of the blood sample from a blood vessel of the patient by means of cannulation. This at least one procedural step can include:

S1: Exchanging data, in particular the registered patient identifier, with a further apparatus of the system, wherein this exchange of data in particular provides for the querying of a database, which is stored on a data storage apparatus of the system or on a data storage apparatus provided external of the system, based on the registered patient identifier so that patient data queried by the control system correlating to the registered patient identifier is drawn from the database;
S2: In particular exchanging data, particularly the registered patient identifier, with an pick-and-place system for patient and/or treatment-dependent equipping of an equipment kit with medical equipment;
S3: Selecting an available or treatment/patient-suited cannulation robot of the system;
S4: Operating a transport apparatus of the system for transporting an equipment kit selected dependent on the patient and/or the treatment, in particular by a sorting apparatus, to at least one cannulation robot;
S5: Selecting the necessary program parameters for performing the automated cannulation.

The system, preferably the control system, in particular a control apparatus of the control system and/or a control device of an apparatus of the system and/or the control procedure is preferably configured to perform, in particular after the start or prior to the end of the cannulation and/or during the withdrawal of the blood sample obtained by the automated cannulation, at least one accompanying parallel procedural step which in particular can be performed at least partially or entirely parallel to the cannulation. This at least one accompanying procedural step can include:
S6: Observing the at least one automatically implemented cannulation by means of an observation device of the system, wherein the observation device can comprise: a camera and/or a microphone and/or at least one sensor for observing at least one physical parameter in the treatment chamber of the cannulation robot accommodating the patient's body part to be cannulated; this physical parameter being, for example, temperature, moisture, atmospheric composition; the observation device can also comprise a device for measuring the success of a disinfecting measure performed on the patient's skin by means of a disinfecting device; the observation data obtained from the observation can be stored in the system or forwarded; in so doing, a physician who is not physically present during the treatment can for example witness the automated cannulation and intervene to stop the automatic cannulation if needed by means of an optionally provided electrical interrupt circuit;
S7: Electronically logging at least one planned process step for the preparation, implementation or post-administration of the automated cannulation, in particular the disinfection, venipuncturing and/or blood withdrawal; the electronic logging can be performed by means of an optionally provided logging device of the system which can in particular comprise a data processor and/or a data storage device; the logging device can be provided separately from the control system as an autonomous device of the system, the logged data of which the system cannot access subsequent logging—thereby providing protocol security and authenticity; the logging device can, however, also be fully integrated into the system, in particular the log data can be stored in the database and correlated with the registered patient identifier and/or date and time of the cannulation; the electronic logging hereby constituting the recording and storing of log data which, for example, can comprise observation data; preferably, the automated cannulation and/or electronic logging are designed such that the automated cannulation of patients complies with one or more legal standards or medical guidelines; in particular, an electronic certificate corresponding to a standardized log data sheet can be issued for each cannulation and output and/or stored, in particular in the database;

The system, preferably the control system, in particular a control apparatus of the control system and/or a control device of an apparatus of the system and/or the control procedure are preferably designed to perform at least one secondary procedural step after the start of the automated cannulation and/or after completion of the cannulation of the at least one blood vessel and/or after removing the blood sample obtained by means of the automated cannulation. This at least one secondary procedural step can comprise:
S8: Applying a fixation tape, in particular a fixation tape selected on the basis of the registered patient identifier, to the skin and/or the cannula inserted into the blood vessel in order to fix the cannula on the skin; the cannulation robot preferably comprising a fixation tape device designed to guide and position adhesive tape for that purpose; the program parameters of the automated cannulation can thereby be selected on the basis of the registered patient identifier, thus individual to each patient, by making use of patient data on the position and/or dimensions and/or condition of the patient's known body part, on the basis of which the configuration and/or dimensions of a suitable fixation tape are selected;
S9: Swabbing the punctured skin site of the cannulated blood vessel with a swab and, in particular, using a swabbing device of the cannulation robot; the swabbing device can comprise a tool arm, its tool designed for holding and/or guiding swab material;
S10: Conducting the blood from the cannulated blood vessel through the cannula in a blood guiding device, in particular to a hemodialysis device or a sample container having been previously selected based on the patient and/or treatment and provided as medical equipment; preferably data-based assigning of the registered patient identifier and a sample container identifier, in particular applying a label on the sample container, e.g. a label containing information printed by means of a labeling device of the cannulation robot;
S11: Transporting a blood sample obtained by the automated cannulation to one or more apparatus, in particular laboratory machines, of the system, in particular to realize a previously specified patient and/or treatment-dependent treatment plan for the analysis, diagnosis or storage of the blood sample; storing the analysis and/or diagnostic data determined by the other apparatus as patient data;

The system, in particular a cannulation robot, preferably comprises one or more of the following apparatus described above and/or further below: equipment holder, in particular equipment box, sorting apparatus, storage apparatus, equipping device, fixation device, measuring device, treatment chamber, vascular structure measuring device, disinfecting device, prepping device, tool arm, blood transport device, sample container, data storage apparatus, pick-and-place system, transport apparatus, fixation tape device, swabbing device, labeling device, other apparatus such as laboratory machines for analysis and/or diagnostics. Further possible and preferential devices as system components are described elsewhere.

The system, in particular the at least one cannulation robot, can comprise an identification system which detects the position and/or dimensions and/or condition (e.g. skin color, skin morphology) of the body part to be venipunctured positioned in the treatment chamber, in particular continuously and/or in real time, by means of a sensor system based on measuring radiation and/or light and/or ultrasound, and stores it in the form of identifying data. The identification system can comprise the vascular structure measuring device.

The identifying data is likewise patient-dependent, however not acquired until after the start of the automated cannulation. The identifying data preferably comprises the patient-based data on the position and/or dimensions and/or condition of the venipunctured body part and/or the position and/or dimensions and/or condition of the subcutaneous vascular system associated with the body part containing the blood vessel to be cannulated, in particular an arteriovenous fistula. The identifying data is not determined until after the automated cannulation has been started and is thus not able to be used in preparing for the automated cannulation. The identifying data is preferably stored in a data storage device, in particular as patient data. It is preferential for the system, in particular the least one cannulation robot, to be designed to prepare the automated treatment by applying historical data, wherein the historical data can be a part of the patient data and can be stored in the database.

The system can comprise a robotic tool arm, which can in particular comprise at least one connection point for connecting a tool head. A tool head is therefore of preferably module design. A tool head is preferably configured to realize a specific function of the cannulation robot. A first tool head can be designed to disinfect the skin by, for example, applying a disinfectant onto the skin, a second tool head can be designed to hold and guide a cannula, a third tool head can be designed to dab a punctured site on the skin using swab material, a fourth tool head can be designed to fix a cannula inserted into the blood vessel by means of fixation tape. A tool arm of this type is also referred to herein as a universal tool arm.

Patient data can be acquired via the registration procedure or another data collection process performed by the system and stored in a database, in particular the patient database, or it can have been stored previously.

Treatment data can be acquired via the registration procedure or another data collection process performed by the system and stored in a database, in particular the treatment database, or it can have been stored previously

Patient data and treatment data can be cross-correlated so that the patient data and treatment data thereby correlated can be clearly determined from a patient identifier, particularly by the system; this correlation information or correlation data respectively can be stored in the database. One database can be provided in which the patient data, the treatment data and the correlation data can be stored.

A “patient identifier” is an identification in data form which preferably identifies a specific patient or a specific group of patients. In particular, the specific patient or specific patient group, to which a specific patient is assigned, can be identified by means of the patient identifier. A patient identifier is in particular a patient number, a patient name, provided it is explicit or can be made explicit by an additional identifier, a (health) insurance number, an ID number or unique number/name of the specific patient group. A respective group of patients in the sense of the invention can in particular be male or female patients, patients with a specific chronic illness, patients having a certain blood type, patients having a certain skin type, dialysis patients with arteriovenous fistula (AV fistula)—i.e. with a Cimino shunt, dialysis patients with vascular graft access (AV graft)—i.e. in particular with a Scribner shunt—or dialysis patients who require a central venous catheter line. The information identifying such a group can also be stored in an optionally provided database of the system in the form of patient data and/or treatment data and can be distinctly cross-correlated with a patient identifier.

A “treatment identifier” is an identification in data form which identifies a specific treatment or a specific group of treatments. In particular, the specific treatment or specific treatment group, to which a specific treatment is assigned, can be identified by means of the treatment identifier. A treatment identifier is in particular a treatment number or a unique designation for a treatment or a group of treatments. A respective group of treatments in the sense of the invention can be specific operations, a specific illness therapy, initial patient examinations or dialysis treatments, which in turn can comprise subgroups, in particular hemodialysis, hemofiltration, hemodiafiltration, hemoperfusion or peritoneal dialysis treatments. The information identifying such a group can also be stored in an optionally provided database of the system in the form of patient data and/or treatment data and can be distinctly cross-correlated with a treatment identifier.

The term “cannulation” refers to a procedure in which a cannula is inserted into the blood vessel in the patient's body part by puncturing the skin and venipuncturing the blood vessel wall so that the distal end of the cannula is disposed in the blood vessel and the proximal end of the cannula is disposed on the outside of the body part so that a fluid connection can be established between the cannula and the blood vessel, by means of which fluid, in particular blood and/or fluid media, can be exchanged via the fluid connection. The “exchange” of fluid in this context means that fluid from the patient's blood circulation is conveyed to an extracorporeal fluid system, i.e. situated external of the patient's body, in particular for fluid storage or fluid conduction, and/or includes conveying fluid from the extracorporeal system into the blood circulation.

A cannula is a tubular body, in particular a rigid or flexible injection needle, with a lumen having a geometry and external dimensions suitable for use in blood vessel cannulation.

The inventive system for data-dependent automated cannulation of patient blood vessels, also referred to as “system” for short in the following, in particular comprises at least one apparatus and at least one method which, particularly in tandem, solve the underlying technical task. Among the at least one apparatus is preferably one or more cannulation robots, preferably at least one control system and preferably one or more user interface devices.

The procedures used in the system preferably include the control procedure, preferably the selection procedure for selecting the program parameters as a function of the registered patient identifier, and preferably a registration procedure which registers a patient in the control system, wherein the result of the registration procedure includes there being a registered patient identifier in the control system.

In the context of the present invention, a control system, or a control apparatus of the control system or a control device of an apparatus of the system generally comprises in particular a data processing device, in particular a data processor, thus a central processing unit (CPU) for processing data, and/or a volatile or non-volatile memory, and/or a microprocessor, or is a data processing device. A CPU of the control system is preferably configured to control the system and in particular to control the cannulation process.

The control system can be formed by a single control apparatus of the system. Preferably, the control system comprises a plurality of control devices which can be independent devices or components of other system apparatus, in particular the at least one user interface device and/or the at least one cannulation robot. In particular, some or all of these control devices can be structured into a data exchange network. In the case of the at least one user interface device comprising its own control device and/or the at least one cannulation robot comprising its own control device, these control devices can be regarded as component parts of the control system. It is however also possible for the control system to not comprise these optionally provided control devices. The control system is preferably designed to execute the registration and the control procedure, in particular by a program code applicably designed for this purpose and executable by a data processor.

The control system of the system and/or the at least one cannulation robot and/or the user interface device—in particular all—can be integrated into one physical device unit, although can also be its own physical device unit. A physical device unit can in particular be a module which at least is or can be data-linked to the system. The control system and/or the at least one cannulation robot and/or the user interface device or component parts of these components can also be at least partly implemented by software functions or can, in particular, partially implement program code. A cannulation robot can comprise a control device, e.g. a computer, which, in combination with software functions, at least partially implements in each case one or more control system functions of the system and/or at least one cannulation robot and/or the user interface device.

A module can in particular comprise the control system or its component parts and/or a user interface device. A module is a separate device from other devices—in particular the apparatus of the system—and/or a separable device from other devices, in particular cannulation robots. A cannulation robot can comprise a connecting device, by means of which the module can be mechanically connected to the cannulation robot, in particular by means of a user-releasable connection. A module can be portable; i.e. can be transported by a user. The module can also be fixedly attached to the cannulation robot. The modular structure offers advantages in the provision of the system. A portable module furthermore provides greater flexibility in the use of the system.

In one preferential configuration of the system, the control system and/or preferably the at least one user interface device are a component part of the at least one cannulation robot. In this case, the at least one cannulation robot preferably comprises the control system and/or the at least one user interface device. In a compact configuration, the system therefore comprises exactly one cannulation robot having the control system and the at least one user interface device.

In the preferential embodiment of the system, which comprises at least two cannulation robots, the control system is preferably a component part of one of these at least two cannulation robots. The at least one user interface device is in this case preferably a component part of the first of the at least two cannulation robots, or is a component part of at least one other second cannulation robot, or forms a separate apparatus or component part thereof. The use of at least two cannulation robots in the system offers the advantage of increased throughput by the at least two cannulation robots operating simultaneously, which is particularly advantageous in the case of systems configured for use in clinical settings.

In addition, the providing of multiple cannulation robots offers the possibility of at least two of the cannulation robots being configured differently in order to be used in different situations. These situations can allow for different types of patients, e.g. the physical size of a patient, in terms of equipment, particularly differentiating between children and adults. Furthermore, these situations can provide for apparatus-specific blood vessel cannulation at different parts of the body, for example by a first cannulation robot being configured for cannulation in an arm and a second cannulation robot being configured for cannulation in a leg. The apparatus adaption can in particular relate to the dimensioning of component parts, e.g. the different dimensioning of a fixation device for immobilizing the body part containing the blood vessel to be cannulated.

A further preferential feature of the system comprising multiple cannulation robots involves at least two of these cannulation robots being positioned at geographically different locations. Automatic cannulation care, which is in particular controlled by a common control system, can thereby be respectively provided at different geographic locations. These different geographic locations can in particular be distributed within a clinic, e.g. in different rooms of one or more buildings and/or on different floors of a building, or within a medical care service area, in particular within a city, or can in principle also be distributed regionally, in particular nationally or internationally. The control system is preferably a central apparatus which controls the different geographically distributed cannulation robots. Multiple control devices of an individual control system can, however, also be distributed, in particular in different geographic locations, or multiple geographically distributed control systems can be provided as component parts of the system.

In particular, the same number of control systems and cannulation robots can be provided in the system. Each cannulation robot can comprise a control system. The cannulation robots are in this case preferably designed for data exchange with at least one other device, in particular an external device.

The cannulation robots are preferably configured to form a data link and/or comprise a data link to an external device. The external device can be a data storage apparatus or a server to which said data storage apparatus is connected. The data storage apparatus preferably contains a database, the data of which can in particular be stored in a non-volatile storage medium of the data storage apparatus. This database can be or contain the patient database.

Preferably, the system comprises a data storage apparatus in which a patient database containing the patient data sets of a plurality of patients is stored, said patient data sets in each case containing at least one patient identifier.

If the system comprises a plurality of control systems, one individual control procedure is preferably configured to be performed utilizing a portion of these control systems or preferably each of these control systems. It is however also possible and preferential for the system to have a plurality of control procedures, in particular multiple copies of a control program for implementing the control procedure for respective execution by a control system.

Preferably, at least two of the apparatus forming component parts of the system are situated at geographically different locations. Preferably, at least one or more of the apparatus constituting component parts of the system are configured as mobile apparatus. A mobile apparatus in the present context is to be understood as an apparatus which, in its intended use, can be and also is positioned at different geographical locations. The dimensions and mass of such a mobile apparatus is thereby adapted such that in intended use, the mobile apparatus can be transported, moved or repositioned under the predetermined effort thereto. Mobile use within the context of the system in particular includes a mobile apparatus of the system being arranged on a means of transport, in particular a transport vehicle, which lends the apparatus the desired mobility.

Pursuant to its intended use, it is particularly preferably provided for a mobile apparatus to be used as a component part of the system while in motion. It can in particular be provided for the means of transport to be a motor vehicle, e.g. an ambulance, ship, train, airplane, helicopter or elevator. A user interface device can be mobile so as to enable patient registration during transport. Furthermore, a cannulation robot can be mobile in order to also enable medical treatment care requiring cannulation to be provided in the vehicle.

Preferably, the at least one user interface device is positioned or positionable at a geographically different location than the at least one cannulation robot. In particular, at least one user interface device is a mobile apparatus. It is also possible and preferential for the at least one user interface device to be a component part of at least one apparatus of the system which can, for example, be an external device, in particular a computer or a server.

Preferably, the system comprises at least two user interface devices, whereby preferably at least two of these user interface devices are situated at geographically different positions.

The data storage apparatus, which preferably stores the database, in particular the patient database, is preferably positioned or positionable at a different geographic location than the at least one cannulation robot and the at least one user interface device. The data storage apparatus can in particular be data-linked to the other apparatus of the system via a network, e.g. a Wide Area Network (WAN) and/or the Internet. The database can be disposed at a central location in the geographically differing positioning and the other apparatus of the system can access the same database via data links. The data storage apparatus can however also be disposed at the same geographical location as a cannulation robot, e.g. within the same room or building or clinic, or can be a component part of a cannulation robot.

An apparatus of the system, preferably each apparatus of the system, preferably comprises one data interface which enables a data link to be formed to another apparatus of the system. Furthermore, an apparatus of the system, preferably each apparatus of the system, preferably comprises a communication device which in particular enables a wireless data link to another apparatus of the system or to a network, in particular a local or global network, particularly the Internet.

The system preferably comprises a network for the exchange of data. Preferably, all the apparatus of the system are configured to exchange data over the network. The network can comprise a central server with which each of the system apparatus are preferably data-linked or configured so as to be able to be connected to form a data link. The network can also be formed by apparatus connected in network-like manner or in ring-like manner. The network can comprise one or more routers and/or hubs via which one or more apparatus of system are connected for exchanging data within the system on the network. The network can in particular implement one or more Ethernet standards. In order to realize the data link, an apparatus of the system, in particular each apparatus of the system, can comprise a data interface device and/or a communication device.

In one preferential configuration of the system, at least one cannulation robot and one user interface device of the system are configured as component parts of an ambulatory treatment station, which is in particular mobile and which can in particular be set up at the residence of a patient. This type of configuration is in particular advantageous for dialysis patients who regularly require frequent cannulation. The patients are spared the effort of visiting a clinic in order to undergo hemodialysis yet nevertheless benefit from the advantages of the invention.

The term “treatment” in particular means a laboratory sample, in particular a blood sample or a volume of blood, being moved and/or transported and/or tested and/or physically, chemically, biochemically or in some other way modified, particularly in terms of its composition.

A communication device is preferably configured to transmit and/or receive data, in particular for data exchange over a data link provided by the communication device, particularly for a remote data link to a remote device. In particular, the device situated remotely from a cannulation robot is also referred to as a “remote device” or external device. In particular, a data processing device or apparatus which is not a component part of a cannulation robot is also referred to as an external data processing device/apparatus. The data link, in particular remote data link, can be established by a restricted (in particular intranet) or global network of computers (in particular a WAN and/or the internet). The data link, in particular remote data link, can also be established by wireless connection, in particular radio link. The data link, in particular remote data link, can in particular be established by mobile radio connection.

A data link connects in particular two data processing units, in particular two data processing devices or apparatus, in a way so as to enable the exchange of data between the units, either unidirectionally or bidirectionally. The data link can be realized in wired or wireless manner, in particular as a radio link. A remote data connection connects in particular two data processing units, particularly two data processing devices, disposed at a distance from one another, thus not being component parts of the same device, in particular the same cannulation robot, user interface device or control system, if the cited devices are realized as separate units. A data link, in particular remote data link, of one device to another device is preferably realized by a direct connection between the two devices or by an indirect connection of the two devices such that a third device is connected between the two devices in order to pass on the data. A remote data link can in particular be realized by a network of computers with which the devices connected by the remote data link are interconnected via the network. The network can be a restricted network, e.g. an intranet, or global network, in particular a WAN and/or the Internet.

A data processing device preferably comprises a central processing unit, in particular a CPU, further preferably at least one data storage device, in particular for the volatile and/or non-volatile storage of data. The data processing device is preferably designed to create one or more first data links to one or more user interface devices, which in particular can be component parts of a cannulation robot, via a first interface device, and preferably create a second data link to a cannulation robot via the second interface device.

An interface device serves the connection of two devices respectively able to process, in particular transmit and/or receive, signals, in particular information, particularly data. An interface device can comprise at least one hardware interface and/or at least one software interface.

Hardware interfaces are in particular interfaces between electrical units as per the usual understanding in electrotechnology and electronics. In the present case, the term “hardware interface” refers in particular also to the connecting components themselves between at least two electrical units, thus in particular all the component parts which contribute to making the connection possible, e.g. the integrated circuits, electronics and lines via which electrical signals are transmitted between the at least two electrical units. Said two electrical units can in particular be a cannulation robot and an external data processing device or two cannulation robots or two electrical units within one cannulation robot. Although not mandatory, a hardware interface can comprise a releasable connecting device, in particular at least one plug, for disengaging and/or restoring the connection.

Software interfaces, in particular software-side data interfaces, are in particular logical points of contact in an information management system, in particular software system: They enable and regulate the exchange of commands and data between different processes and components. Software interfaces can be data-oriented interfaces used only for communication. In this case, a software interface only contains the information to be exchanged between participating system components.

A—in particular external—data processing device or data processing apparatus can be a computer, in particular a server, which is designed in particular to create a data link to more than one user interface device and/or more than one cannulation robot. A—in particular external—data processing device or apparatus can comprise a computer or a microprocessor or can be a computer or microprocessor. A server is in particular a computer, the hardware of which is adapted to server applications. An external data processing device can be a mobile data processing device designed to establish a wireless data connection, in particular a data link, over a restricted computer network, in particular an intranet, or a global computer network, in particular the internet. A computer network is a consortium of different technical, primarily independent, electronic systems (in particular computers but also sensors, actuators, agents and/or other functional components, etc.), which enable the individual systems to communicate with one another.

The control system is preferably designed to clearly differentiate between different patients or users. The control system preferably clearly identifies a patient or user. To that end, the control system preferably processes identifying data. Preferably, the access control is designed to authenticate registered patients or users; i.e. perform a verification process which checks the authenticity of the requesting patient or user and authenticates the patient or user upon positive verification. Authentication data comprises, for example, a registration text and a password text or a data set on facial recognition or an iris scan or fingerprint scan or other data or biometric data. Biometrics offers the advantage of the patient or medical personnel being able to establish identity without cognitive effort, which is particularly advantageous when, for example, the patient is unresponsive or is unable to actively take part in the authentication for other reasons. The authentication can furthermore be performed by means of RFID chip or NFC chip or by gesture recognition. An authentication can in particular ensue by directly accessing, locally or remotely, the laboratory machine or its access control respectively.

The control system and/or the control procedure can in addition be designed to register the registering patient as a function of successful system authentication, ascertain a patient identifier and/or recognize the patient as the registered patient by identifying the registered patient identifier. The authentication can require the patient to have preregistered with the system so that the system has comparative data available for the identification. The control system and/or the control procedure can be designed to register a patient in the system, whereby a clearly attributable record of patient data including the patient identifier is associated with the patient. This patient data set can be stored in the patient database.

In addition, a patient can also use an identifying medium, for example a patient card, identity card or other clearly identifiable document able to be identified by the system's user interface device. A user interface device preferably comprises an authentication device which can comprise an information reader. The control system and/or the control procedure can respectively be designed to register a clearly identifiable document of the patient via the system's user interface device by the user interface device in particular comprising an information reader, e.g. an optical scanner, a magnetic strip reader or an RFID chip reader. An information reader can in particular be designed to read coded information or be designed as a biometric scanner. A biometric scanner can be designed e.g. as a fingerprint scanner or an iris scanner.

The system preferably comprises an information management system, with which the registration of the patient in the system is realized.

Each user can establish a first data link to the system via the same user interface device or multiple users can establish a first data link to the system via different user interface devices. A user interface device can be a component part of a cannulation robot. A user interface device can be a component part of an external device.

A user interface device in each case preferably comprises: a control device for the user interface device; a communication device to establish a data link with a cannulation robot and/or the control system via an interface device of same; an input device for recording user inputs; an output device, in particular a display, to output information to the user.

The control device of the user interface device is thereby preferably designed to exchange data obtained from the user inputs with the control system and/or the cannulation robots via the data link and which effect the inventive cannulation robots employed to register the user of the control system, in particular the control procedure, as the registered user, wherein particularly in consequence of this control system registration, the individually assigned patient identifier, referred to as the registered patient identifier, is used for the registered patient.

The term “machine-controlled treatment” of a laboratory sample, in particular a blood sample or a volume of blood, means that the treatment of the at least one laboratory sample is at least partly controlled, in particular performed, by the machine, in particular a laboratory machine. To the extent the treatment is controlled and/or performed by the machine, it is inasmuch in particular not controlled and/or performed by the user, particularly not manually controlled and/or performed by the user.

Machine-controlled treatment is preferably to be further understood as the treatment being at least partly controlled, in particular performed, by the machine, in particular laboratory machine, as a function of a patient identifier, in particular the registered patient identifier. The user registration input can occur prior to the start of the treatment on a laboratory sample of a patient and/or during said treatment. The user input preferably occurs via the user interface device, which is preferably a component part of a machine or cannulation robot, or is preferably provided separately from the cannulation robot and is signal-connected to the control system. The user input serves in particular the inputting of at least one parameter, its value influencing and/or controlling the cannulation by the cannulation robot and/or the treatment by a machine or laboratory machine. This parameter can in particular be a program parameter.

The “machine-controlled treatment,” which can in particular be part of a “machine-controlled cannulation,” designates in particular at least semi-automated or automated treatment. In semi-automated treatment, it is in particular possible for the treatment to be carried out such that there is at least one user input after the start of the treatment and prior to the end of the treatment by means of which the user, e.g. medical personnel, can influence the current treatment, in particular by, for example, responding to an automatic prompt on a user interface device, in particular confirming or rejecting an input or entering a different input. In semi-automated treatment, it is in particular possible for the treatment to comprise a plurality of treatment steps which are in particular automatically performed in close succession and comprise at least one treatment step requiring a user input to be made, in particular via a user interface device. In automated cannulation by a cannulation robot, it can preferably be provided—and a cannulation robot correspondingly configured—for the automated cannulation to be contingent on the user input.

It is preferably provided for the automated cannulation to not start until the patient himself enters a user input, by means of which he starts the cannulation. Or it can also be provided for the automated cannulation to not start or to be stopped when the patient himself enters a user input. The system can in this way be designed such that the registered patient is given safety instructions, patient rights and/or other information, e.g. via the user interface device, before the automated/semi-automated cannulation will start or continue. It can thereby be guaranteed that the patient is prepared and can be safely cannulated.

A machine-controlled treatment, in particular cannulation, is preferably a program-controlled treatment; i.e. a treatment controlled by a program. Program-controlled treatment refers to the treatment process substantially ensuing by means of multiple or a plurality of program steps being executed. Preferably, program-controlled treatment ensues based on the use of at least one program parameter, in particular at least one user-selected program parameter. A parameter selected by a user is also referred to as a user parameter.

Program-controlled treatment preferably ensues with the aid of a data processing device which can in particular be a component part of the control system or the control device of a cannulation robot. The data processing device can comprise at least one processor; i.e. a CPU, and/or at least one microprocessor. The program-controlled treatment is preferably controlled and/or performed in accordance with the specifications of a program, in particular a control program. In particular, substantially no user interaction is necessary in a program-controlled treatment, at least after the necessary user-side program parameters have been registered.

A program parameter is to be understood as a variable which can be set in a predefined manner within a program or subroutine for at least one execution (call) of the program or subroutine. The program parameter is specified, e.g. by the user, and controls the program or subroutine and effects a data output as a function of said program parameter. In particular, the program parameter and/or the data output by the program influences and/or controls the controlling of the machine, in particular the controlling of the treatment, in particular cannulation, by means of at least one cannulation robot or machine, in particular comprising an automated or semi-automated treatment device.

A program parameter can be a program parameter required from the user. A required user-supplied program parameter is characterized by being required in order for a treatment, in particular the cannulation, to be performed, e.g. a user input, this starting the cannulation and/or the venipuncture procedure. Other program parameters, which are not required from the user, can be derived or otherwise made available from the required user-supplied program parameters, in particular optionally set by the user. The setting of a program parameter by a user ensues in particular from the display of a selection of possible predefined values from a list of predefined values stored in the system or in the cannulation robot or laboratory machine, wherein the user selects and thus sets the desired parameter from this list. It is also possible for this program parameter to be set by the user by entering the value corresponding to the desired value by, for example, entering the number into a numeric keypad or by the user continuously or incrementally increasing or decreasing a value until reaching the desired value and the value thus set. Other forms of input, e.g. language control and/or gesture control, are conceivable.

A program refers in particular to a computer program. A program is a series of instructions, in particular consisting of declarations and directives, enabling the executing and/or resolving of specific functions, tasks or problems on a digital data processing system. A program is usually provided as software employed with a digital data processing system. The program can in particular be firmware; in the case of the present invention, in particular as firmware of the control system or the control device of a cannulation robot or laboratory machine and/or user interface device. The program is usually provided on a data medium as an executable program file, frequently in so-called object code, which is loaded into the main memory of the computer of the digital data processing device in order to be executed. The program is processed by the processors of the computer as a succession of machine, i.e. processor, commands and thereby implemented. A “computer program” in particular also refers to the source text of the program from which the executable code can be taken during the process of controlling the laboratory machine.

An instruction is to be understand in the customary manner as a central element of a programming language. The programs of such languages consist primarily of one or more instructions. An instruction represents an individual directive formulated in the syntax of a programming language which is to be executed in the context of executing the program. The specific syntax of an instruction is defined by the respective programming language or its specification respectively. In machine-oriented programming, instructions are frequently also referred to as commands. Instructions are usually assignments, control instructions (such as hops, loops and conditional commands) and procedure calls; also to some extent, depending on the programming language, confirmations, declarations, class and function definition instructions. The instructions of the control program can be configured in the usual way.

The control procedure can in particular be designed as a control program in the system which executes the control procedure. A control program refers to an executable computer program which preferably controls and/or implements the desired treatment, in particular cannulation, in particular as a function of at least one program parameter and as a function of the registered patient identifier. This program parameter can be a program parameter influenced and/or set by the user. The treatment, in particular cannulation, can be controlled by the control system or a control device of the cannulation robot or laboratory machine generating one or more control parameters as a function of the program parameters, by means of which for example the at least one treatment device of the laboratory machine or cannulation robot, e.g. a tool arm, is controlled or its kinetics influenced.

Preferably, the system and/or a cannulation robot and/or a laboratory machine comprises an operating system which can be or comprises a control program. The control program can in particular indicate an operating system of the system and/or the cannulation robot and/or laboratory machine or a component of the operating system. The operating system controls the treatment, in particular cannulation, particularly the registration of the patient, and in particular further operational functions of the system. The control procedure can be or comprise an operating system. The control program which preferably implements the control procedure can be run on the operating system.

Preferably, a cannulation robot, in particular a laboratory machine, comprises a communication device for establishing a remote data connection for data exchange with other apparatus of the system or with an external device which likewise has a suitable communication device for establishing a remote data connection for data exchange with the cannulation robot or laboratory machine. Such a communication device can be configured to form a radio link, in particular a mobile radio connection. Preferably, the communication device is thereby equipped to enable remote access to the cannulation robot or the laboratory machine by a user, in particular the selecting or setting of at least one parameter, in particular a parameter which controls a function of the cannulation robot or the laboratory machine, in particular the function of implementing cannulation or other treatment.

The term laboratory machine refers in particular to a machine designed for the machine-controlled treatment of at least one laboratory sample and which is designed for use in a laboratory. Said laboratory can in particular be a chemical, biological, biochemical, medical or forensic laboratory. Such laboratories serve in the research and/or analyzing of laboratory samples, although can also serve in the production of products using laboratory samples or the production of laboratory samples. It is preferred that the cannulation robot not be understood as a laboratory machine. A cannulation robot can be understood as a representative of a new genre of machines which can be called at least partly or fully autonomously operating medical robots. The inventive system is to be categorized in the genre of such at least partly or fully autonomously operating medical robots. If at least one or more cannulation robots of the system are used in a clinical setting, and in particular based there, this constitutes an at least partly or fully autonomously operating clinical treatment system. This is a preferential type of invention embodiment. The same applies to the inventive method.

A laboratory sample is a sample which can be processed in a laboratory. A laboratory sample is in particular a blood sample obtained by cannulation, in particular a volume of blood. Instead of the term laboratory sample, the description of the invention also uses the term “sample.” A liquid sample can be one as is usually handled in a biological, chemical or medical laboratory. A liquid sample can be an analysis sample, a reagent, a medium, a buffer, etc. A solution comprises one or more solid, liquid or gaseous substances in solution (solute) and further comprises a preferably liquid solvent (solvent) in particular constituting the greater or greatest portion of the volume forming the solution.

The solvent can itself be a solution.

The—particularly automated or semi-automated—treatment of a laboratory sample (samples), in particular a blood sample obtained by cannulation, can comprise one or more of the following specified processes, in particular concurrently or successively:
Transporting the laboratory sample, in particular by means of a transport device, using the effect of gravity and/or a force effected by the cannulation robot or a laboratory machine;
Physically treating, in particular thermally treating, the sample, without contact (non-invasively); or freezing or thawing the sample; optically treating the sample; moving the sample, thus effecting a mechanical treatment of the sample, particularly agitation, centrifuge;
Chemically, biochemically or biomedically treating the sample: Adding chemical, biochemical, or biomedical (blood, serum, cell medium) substances;
Storing the sample, in particular for a program-controlled defined period, in particular under specific physical conditions, e.g. at specific temperature, and/or setting a defined environmental atmosphere, e.g. an inert gas or a specific air humidity, under specific radiation conditions, e.g. shielded from visible light, in darkness or at a defined irradiation;
Measuring or analyzing the sample, in particular analysis by non-invasive and/or invasive treating of the sample, in particular for the purpose of measuring at least one or more chemical, physical, biochemical and/or medical properties of the sample; in particular cell counts by means of a cell counter;
Performing a diagnostic procedure on the sample in order to in particular identify or exclude a pathogenic patient state;
Processing the sample, in particular modifying at least one property of the sample, in particular by non-invasive and/or invasive treating of the sample;
Performing hemodialysis on a volume of blood, preferably with ensuing return of the blood volume into the patient's blood circulation.

This treatment is in particular effected under program control employing at least one program parameter.

The treatment in particular ensues pursuant to at least one control parameter which determines the treating of the laboratory sample by the treatment device. A control parameter can define a period of time, a point in time, a specific sample volume and/or dosing volume, a specific sample temperature, etc. A control parameter can relate to the automatic use of a specific transport head, a specific type of transport container, a specific type of sample container, one or more individual samples or specific positions of these components in the given workspace. A control parameter can relate to the treating of an individual sample or the treating of multiple or many samples.

Preferably, a control parameter is selected automatically by the laboratory machine, in particular laboratory robot, as a function of at least one program parameter, in particular automatically as a function of the program parameters selected by the user and in particular as a function of a patient identifier or registered patient identifier. This provides the advantage of not needing to individually specify all of the control parameters. A control parameter can also correspond to a program parameter.

The transporting of a sample can be a transporting from a cannula or a tube or a sample container into a transport container and/or from the transport container into a sample container or other target location. This transport is in particular program-controlled using at least one program parameter.

The transport container preferably consists partially or completely of plastic. It is preferably a consumable article which is typically only used for one treatment or a low number of sample treatment steps. The transport container can, however, also consist partially or completely of another material.

The sample container preferably exhibits a sample container identifier. This can incorporate the patient identifier or can be clearly correlated with the patient identifier in the system in order to be able to unambiguously deduce the patient associated with a filled and system-registered sample from the sample container identifier. The blood sample obtained by means of the cannulation robot is preferably stored by the system in at least one sample container; the blood sample is associated with at least one clear sample container identifier in a database of the system, wherein a sample container clearly differentiates a sample container such that each sample container containing a blood sample is unambiguously linked to the patient identifier and the information about the at least one sample container identifier association is stored in the database of the system.

The transport of a sample can be a transport of the sample from an initial position to a target position. The initial position can be when the sample is disposed in a first sample container and the target position of the sample can be its location in a second sample container into which the sample is to be transferred. In the present case, this type of transport is also called sample transfer or transfer. In practice, a sample transfer is usually carried out in order to transfer a sample from one storage container, in which the sample was for example stored and/or which can for example hold a larger volume of the sample, into a second sample container in which the sample will be processed further. This transport is in particular program-controlled using at least one program parameter. The transport of the sample is preferably a transport starting from the cannulation robot, where the blood sample was obtained, to a machine, in particular laboratory machine, where the sample will be processed further.

The transport container is connected or connectable to a transport device of the system, by means of which the sample preferably starting from the cannulation robot, where the blood sample was obtained, is preferably automatically conveyed to a machine, in particular a laboratory machine, where the sample will be processed further. The transport device is preferably a component of a robotic transport system which is preferably a component part of the inventive system.

A sample container can be a single container containing only one sample or can be a multi-container in which multiple single containers are in linked arrangement.

A single container can be an open container or a closable container. In the case of a closable container, a cover element, in particular an end cap, can be provided. The cover element can be permanently fixed to the container, e.g. as a hinged lid or hinged end cap, or it can be employed as a separate component.

A sample container can comprise an information field able to contain information about the sample container or its contents, in particular the sample container identifier. The information field can exhibit coded information, e.g. a barcode, QR code, RFID chip or other coded information. The information can comprise specifics for identifying the sample and/or a sample container. The system can comprise an information reader in order to read this information and preferably provide same to the control system.

The at least one cannulation robot, in particular a laboratory machine, in particular laboratory robot, can be connected or connectable to an LIMS. LIMS stands for Laboratory Information and Management System. An LIMS is a software system in the usual sense related to automated or semi-automated data processing in chemical, physical, biological, medical laboratories. Such data can stem from the measuring of samples and/or can relate to controlling the processing of the data. An LIMS preferably serves in acquiring and evaluating measurement values. An LIMS is used to increase operational performance in a laboratory and/or optimize efficiency in processing laboratory samples.

The cannulation robot, in particular a laboratory machine or laboratory robot, can comprise an information reader for reading information on a sample and/or a sample container and/or a treatment instruction for the sample and/or sample container and, preferably, provide same to the control device of the cannulation robot, the laboratory machine or the laboratory robot.

The cannulation robot, in particular the laboratory machine or the laboratory robot, preferably comprises at least one timer device and/or preferably one timing device, to enable time-dependent cannulation and/or sample treatment. Time-dependent treatment is preferably program-controlled, and in particular controlled by means of at least one program parameter. The chronological sequence to the automated cannulation and preferably subsequent—in particular repeated—treatment can in this way be controlled and/or logged and/or system-coordinated.

The cannulation robot or laboratory machine preferably comprises a user interface device for a user to input data and for displaying information, in particular information contained in said data, wherein the user interface device comprises a display device, in particular a display, particularly a touchscreen display.

The invention further relates to a method for data-dependent automated cannulation of patient blood vessels, in particular for hemodialysis, comprising the following steps:—Recording a registered patient identifier upon a patient registering at a user interface device which is data-linked to a data processing control system;—Determining program parameters as a function of the registered patient identifier by the control system; and—Controlling a cannulation robot set up for the automated cannulation of patient blood vessels and which is data-linked to the control system as a function of the program parameters for automated cannulation of a patient's blood vessel based on the patient's registered patient identifier, whereby in particular the at least one cannulation robot is configured to automatically perform a first venipuncture for insertion of a first cannula into a blood vessel for automatic withdrawing and routing of the blood to a blood guiding system.

Preferably, the inventive method provides for the step of automatically performing a second venipuncture for insertion of a second cannula into a blood vessel for automatically returning the blood from the blood guiding system for performing hemodialysis.

Preferably, the inventive method provides for the step of automatically implementing a selection process for selecting the program parameters to be used for the automated cannulation as a function of the registered patient identifier. Preferably, the selection process provides for the step of accessing a data matrix in which the necessary program parameters are linked based on treatment data and thereby determinable, wherein the system comprises a data storage apparatus (90) in which the data matrix is stored.

Preferably, the inventive method provides for the step of implementing at least one preparatory procedural step after the recording of the registered patient identifier and prior to the start of the automated cannulation as a function of the registered patient identifier which prepares the automatic withdrawal of the blood sample from the patient's blood vessel by means of the cannulation.

Preferably, the inventive method provides for the step of implementing at least one accompanying procedural step during the withdrawing of the blood sample obtained by the automated cannulation as a function of the registered patient identifier which in particular is performed at least in part or entirely parallel to the cannulation.

Preferably, the inventive method provides for the step of implementing at least one secondary procedural step after the start of the automated cannulation and/or after the completion of the cannulation of the at least one blood vessel and/or after withdrawal of the blood sample obtained by means of the automated cannulation as a function of the registered patient identifier.

Preferably, the inventive method provides for the step of determining the location and/or dimensions and/or condition (e.g. skin color, skin morphology) of the body part to be cannulated positioned in the treatment chamber by means of an identification system via a sensor system based on measuring radiation or light and/or ultrasound, in particular continuously and/or in real time, and in particular storing it in the form of identifying data.

Preferably, the inventive method provides for the step of performing an automated treatment by utilizing historical data containing the stored identifying data as a function of the registered patient identifier.

Preferably, the inventive method provides for the step of positioning the at least one user interface device at a different geographical location from the at least one cannulation robot.

Preferably, the inventive method provides for the step of positioning a data storage apparatus at a different geographical location from the at least one cannulation robot and from the at least one user interface device.

Preferably, the inventive method provides for the step of the registration of registering patients as a function of successful system authentication, determining a patient identifier and/or identifying the patient as the registered patient by ascertaining the registered patient identifier.

Further conceivable preferential configurations of the method according to the invention can be deduced from the description of the inventive system and its preferential configurations.

Further preferential configurations of the system according to the invention and the method according to the invention are yielded by the following description of example embodiments in conjunction with the figures and their description. Unless expressly specified or contextually indicated otherwise, the same reference numerals are substantially used to identify equivalent components in the example embodiments. Shown are:

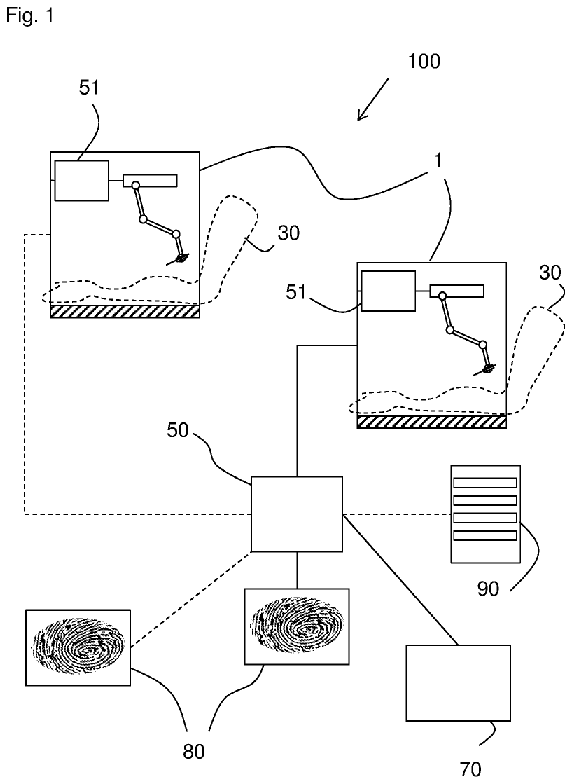

FIG. 1 a schematic representation of an example embodiment of the system according to the invention.

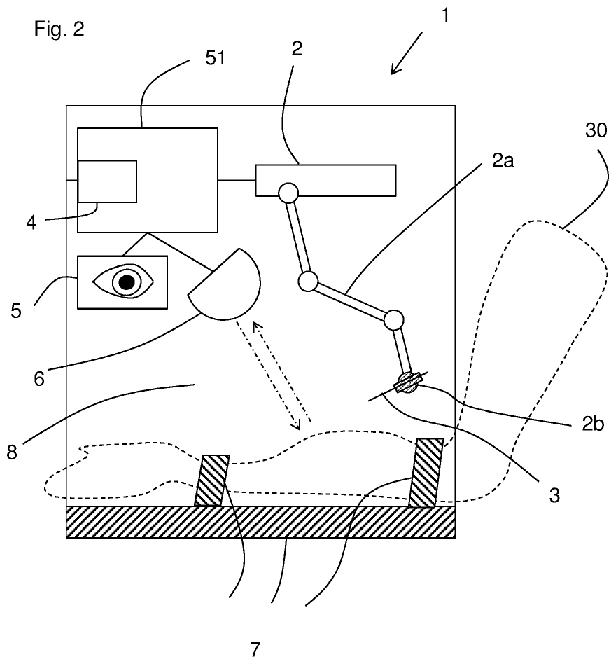

FIG. 2 a schematic representation of an example embodiment of the cannulation robot applicable to the inventive system.

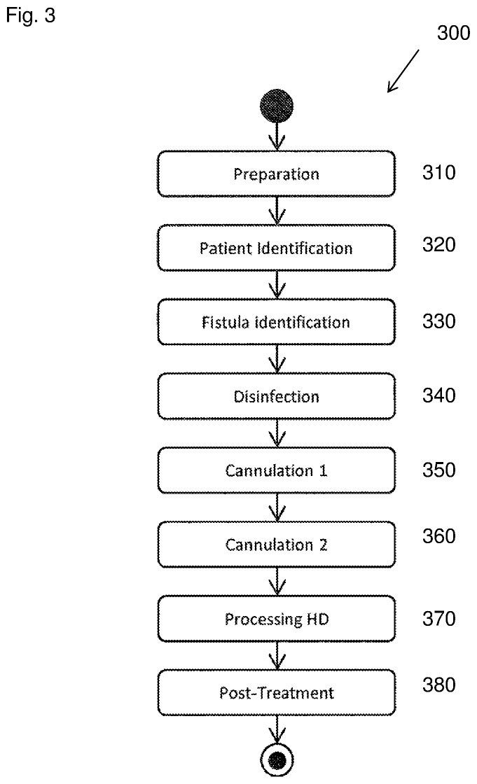

FIG. 3 a schematic representation of an example embodiment of the inventive method or the operative control procedure in the inventive system respectively.

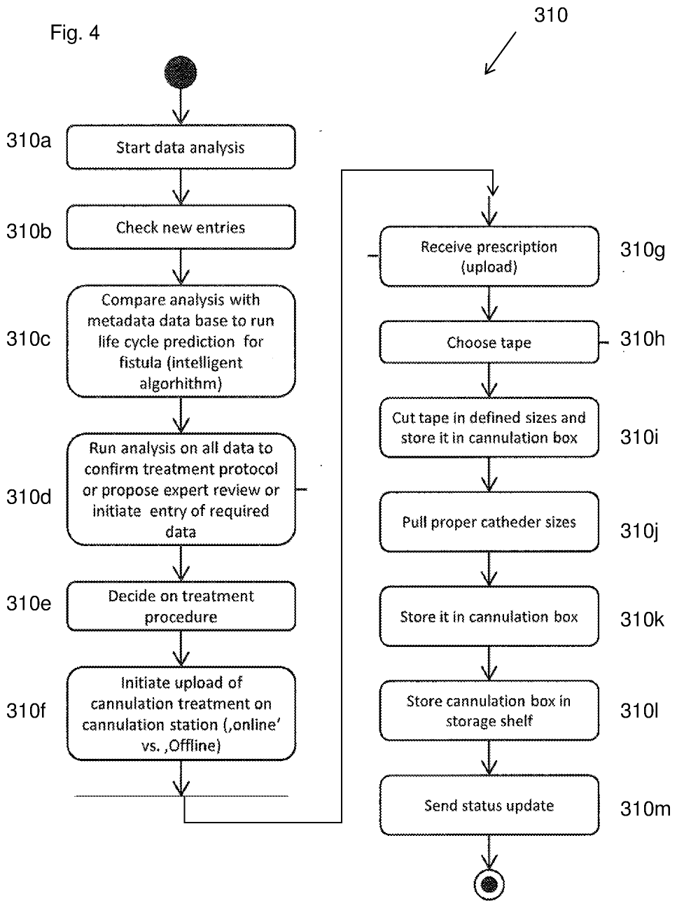

FIGS. 4 to 11 show example embodiments of the substeps of the FIG. 3 method.

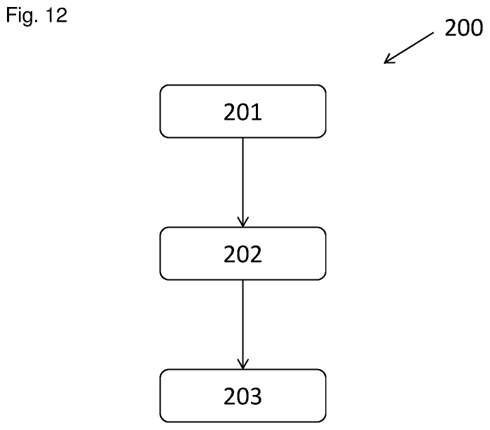

FIG. 12 shows an example embodiment of the inventive method or the operative control procedure in the inventive system respectively.

FIG. 1 shows a clinical system 100 for data-dependent automated cannulation of patient blood vessels in preparation for hemodialysis as an example embodiment of the inventive system. The system 100 comprises two cannulation robots 1, each configured for the automated cannulation of patient blood vessels. A control system 50, 51 of the system 100 is designed to implement a control procedure able to control each of the two cannulation robots as a function of program parameters. The control system 50, 51 comprises a central control apparatus 50. Control devices 51 in each case control a cannulation robot 1 and are regarded as component parts of the control system 50, 51, its task in the clinical system 100 comprising the implementing of the at least one automatic cannulation of a patient's body part 30 as a function of the patient's patient identifier. The control apparatus 50 and the control device 51 each have a data processing device with a data processor and a data storage as well as a communication device for establishing a data link to another communication device of the system. The data links of the apparatus 1, 80, 90 to the control device 50 create a networking of the system components into a data exchange network. In the present case, the network comprises the control apparatus 50 as the central computer.

The system 100 comprises two user interface devices 80, implemented in the example as authentication devices 80 based on a fingerprint scanner, by means of which patients previously registered in the system are authenticated. The registration of a patient comprises collecting data relevant to the clinical admission, in particular storing an image of his fingerprint in a patient database for comparison and assigning an intrasystem patient identifier to the patient data of the patient, referred to here as the patient identifier. After the registered patient has registered, the control system recognizes the patient as the registered patient, his patient identifier is referred to as the “registered patient identifier” subsequent registration.

The system 100 comprises a data storage apparatus 90, which can be a hard disk server, in which at least one database is stored. In particular, the patient database which the system accesses on the basis of the registered patient identifier in order to read the necessary patient data of the registered patient for the automated cannulation is stored in the data storage apparatus 90. The patient data can be regularly supplemented by data collected by the system, e.g. identifying data of an identification device, as well as by optional observation data on the course of a completed cannulation or hemodialysis respectively as well as data on the treatment performed. A patient data set thus results, its accuracy enabling subsequent treatments of the patient in the clinical system to be continuously improved. In this example embodiment, the patient data in particular comprises data on the hemodialysis to be performed on the patient. This patient data in particular comprises vascular structure data, which will be described below.

The control apparatus 50 implements the control procedure of the system, this being in the form of program code executable by the data processors. The control procedure controls the at least one cannulation robot as a function of program parameters, and selects to that end appropriate, patient-dependent program parameters; i.e. subject to the registered patient identifier, subsequent patient registration and prior to the start of the cannulation. The control procedure performs to that end a selection process which is stored in the system.

The selection process is designed to access a data matrix which is stored in the data storage apparatus 90 and in which the necessary program parameters are linked based on patient data and treatment data and thereby determinable. Upon the registration of the patient, the system can realize that only one hemodialysis procedure is to be performed on said patient based on its own stored schedule or based on user input during registration. This information can be known as treatment data. Depending on this treatment data, the system can determine which patient-specific conditions and data in the patient data set are to be observed during hemodialysis.

Patient-specific conditions and data for hemodialysis can include vascular structure data, the patient-specific required type and size of fixation tape and swabbing material or disinfectant, data on the type of cannula to be used in the cannulation, etc. The data matrix can additionally contain information on the “vascular structure data,” “type and size of fixation tape and swabbing material or disinfectant” and “type of cannula to be used in the cannulation” program parameters needing to be observed for the “hemodialysis” treatment. The system then automatically prepares the automated cannulation as a function of these patient-specific parameters by relaying the “type and size of fixation tape and swabbing material or disinfectant” and “type of cannula to be used in the cannulation” program parameters to a pick-and-place system for medical equipment so that the pick-and-place system 70 of the system can furnish the required equipment in a standardized equipment box and by relaying the vascular structure data from the patient data set corresponding to the “vascular structure data” program parameter to the cannulation robot selected by the system for cannulation or instructing the cannulation robot to access said vascular structure data.

Apart from hemodialysis-specific information, the patient data set can also contain information required for other treatments and which is likewise patient-specific, e.g. information on patient medication to be periodically dispensed by the system.

In the example embodiment of the clinical system, the apparatus 1, 50, 80, 90 are in part arranged at different geographical locations. A first group of system apparatus can be disposed within the same building complex of the clinic. This includes the apparatus 1 and 80 connected to the control apparatus 50 in FIG. 1 by the continuous lines. The data storage apparatus 90 preferably allocated to the inventive system can be arranged at a clearly separate geographic location from these system apparatus, e.g. in another building. The data storage apparatus 90 can, however, also be used by other clinical systems. As suggested as a′ connection by the dotted line, a further user interface device 80 and a cannulation robot 1 can be disposed at a further geographically different location, e.g. in an ambulatory hemodialysis treatment station which, for example, can be based at the residence of a dialysis patient. This further user interface device 80 and cannulation robot 1 can likewise be mobile, e.g. in an ambulance, train or ship, provided a data link to the system is maintained.

FIG. 2 shows an example embodiment of a cannulation robot 1 used by the system 100 according to the invention.

The cannulation robot 1 comprises a robot-controlled tool arm device 2 which has a jointed tool arm 2a connected at one end to the cannulation robot 1 and supporting a tool head 2b on the other end. The tool head is realized in the present case as an exchangeable function-specific module subject to the type of treatment, e.g. cannulation, applying fixation tape or swabbing. In the figure, the tool head is configured for venipuncture, or cannulation respectively, and holds a cannula 3 to be inserted into the blood vessel in the arm 30 of the patient. A further tool head (not shown) holds the disinfecting device for the program-controlled application of disinfectant at the site of the patient's skin to be cannulated.

The cannulation robot 1 comprises an observation device 5 which in the present case has a camera, by means of which the automated cannulation procedure can be observed in real time by an observer at a remote location. The video data from the cannulation is additionally or alternatively stored in the patient data storage.

The cannulation robot 1 comprises an identification device 6, with which the position and dimensions of the patient's arm 30 and the position and structure of the patient's subcutaneous blood vessels as well as the morphology of the skin and the blood vessels can be detected. To this end, the identification device 6 comprises a vascular structure measuring device (not specifically shown) which is configured to produce at least one image of the blood vessels by means of ultrasound. These measurements can in particular occur prior to the cannulation and/or during and/or after the cannu-lation. The vascular structure data produced by the vascular structure measuring device can be stored as patient data and furnished in subsequent treatments as historical data, which can improve the cannulation in patient-specific manner.

The vascular structure measuring device can be further configured to detect prior puncture sites on the skin and/or blood vessel. The control procedure can use this information particularly with dialysis patients in order to implement and/or proceed with the cannulation pursuant to the rope ladder technique or buttonhole technique. Buttonhole technique information for the cannulation robot comes from the patient data. In the buttonhole technique, the cannulation robot always inserts the cannula at the same angle relative to the blood vessel, or arteriovenous fistula respectively, at the same position and through the “buttonhole” channel. A robot is destined to provide consistent quality in executing this buttonhole technique, which is crucial to the success of the technique. The buttonhole technique is therefore a particularly preferential type of procedure for performing the cannulation employing a cannulation robot or, respectively a control procedure. The robot also offers advantages in the case of the rope ladder technique compared to manual cannulation sirice, on the one hand, the blood vessel morphology in the vascular structure can be determined and known, whereby the control procedure can select an optimal next site at a distance from the previous puncture and, on the other hand, cannulation can ensue without any vibration at maximum precision.

Possible procedural steps of the cannulation robot, in particular cannulation robot 1, and steps of the control procedure or the inventive method respectively can be deduced from the previously described process steps Px, in particular P1 to P8, and process steps Sx, in particular S1 to S11.

FIG. 12 shows a first general example embodiment of the inventive method 200 used in the previously described system 100. The inventive method in principle preferably provides for the implementation of the process steps also applied in the system with the control procedure. The process steps comprise Step 201:—Recording a registered patient identifier by a patient registering at a user interface device which is data-linked to a data processing control system; Step 202:—Determining program parameters as a function of the registered patient identifier by the control system; and Step 203:—Controlling a cannulation robot set up for the automated cannulation of patient blood vessels and which is data-linked to the control system as a function of the program parameters for automated cannulation of a patient's blood vessel based on the patient's registered patient identifier.

FIG. 3 shows an overview of a further example embodiment of the inventive method 300, here for performing dual cannulation of an arteriovenous fistula for a hemodialysis, in particular employing an example embodiment of the inventive system, e.g. system 100. The method 300 provides for the following sequence of steps in this order:
Step 310: Preparing the automated cannulation
Step 320: Registration with patient identification, preparation
Step 330: Analyzing historical vascular structure data and/or detecting current vascular structure data of the arm 30 inserted into the treatment chamber 8, for planning the cannulation
Step 340: Automatically disinfecting the skin area to be punctured
Step 350: Automatically performing the first venipuncture and cannulation
Step 360: Automatically performing the second venipuncture and cannulation
Step 370: Performing the hemodialysis
Step 380: Automatic post-treatment of punctured site by pressure, swab, gauze

Step 320 ensues alternatively and preferably before step 310.

FIG. 4 shows an example embodiment of preparatory step 310 (preparation):

The system and/or the control procedure begins with the analysis of the task (310a), which yields for example from the patient data of the registered patient in combination with a treatment plan stored in the system. Alternatively, the system is provided the information on the plan, in particular as treatment data on a planned hemodialysis at a specific time, during/prior to/or subsequent patient registration.

The system checks whether database entries already exist with respect to the planned hemodialysis and compares them to the treatment plan (310b). The system determines the vascular structure data from the patient database on the (registered) patient and calculates the possible venipuncture sites using an applicable algorithm (310c). All the data is analyzed and measures derived therefrom, in particular whether a physician needs to be consulted or whether automated cannulation with subsequent hemodialysis is authorized (310d).

The system decides the exact sequence of the process steps for the automated cannulation with ensuing hemodialysis (310e). The system transmits data on needed equipment to the pick-and-place system (310f). The system checks whether the availability of the requested equipment, in particular also the availability of the desired cannulation robot, is automatically confirmed (310g). The system in particular selects a suitable fixation tape for the later fixation of cannulas on the arm (310h). The fixation tape is in particular portioned and sized by the pick-and-place system into patient-dependent and treatment-dependent sections and transferred into a standardized equipment box automatically operated by the cannulation robot (310i). Appropriate catheters and/or cannulas are selected (310j) in patient-dependent and treatment-dependent manner and provided in the equipment box (310k). The equipment box is automatically transported to the cannulation robot (310l). The system sends a status update to the cannulation robot (310m).

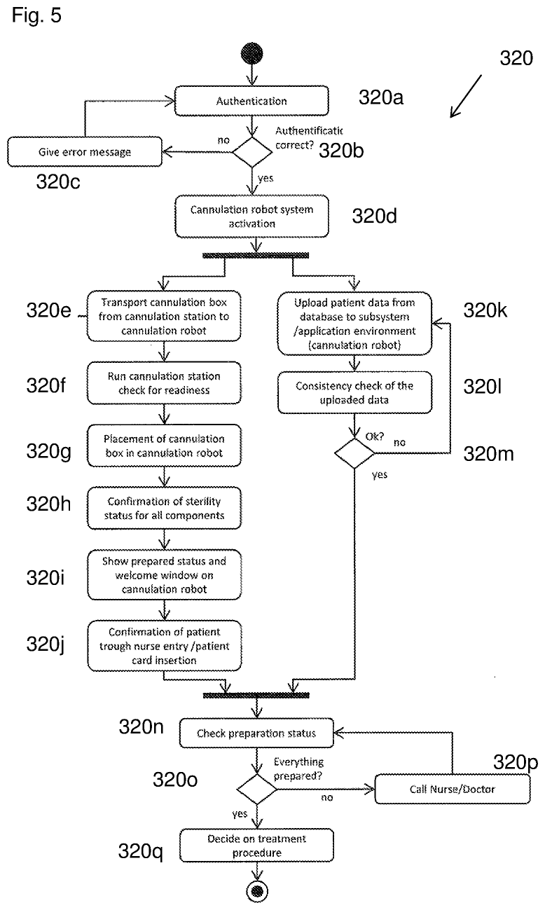

FIG. 5 shows an example embodiment of preparation step 320 (registration, authentication, with subsequent preparation of the clinical treatment):

The system authenticates the patient (320a). this can ensue by means of password, pin or pattern, chip card, FRID chip, SIM card with mTan procedure, imprinted digital certificate or 2D code, TAN/iTAN, preferably using biometrics. If the authentication fails—which can occur if the patient is not yet registered—the system acknowledges with an error message; registration was previously described above and is its own procedure. Upon registration, the system learns of the pending treatment—in this case, a hemodialysis. Directly after registration, the system preferably immediately begins automatically preparing for the hemodialysis, whereby no time is lost, which can be critical in an emergency. In particular, the pick-and-place system for medical equipment is activated (320d).

The automatically equipped patient-dependent equipment box is transported from the pick-and-place system to the cannulation robot (320e). The pick-and-place system transmit a status report (320f). The equipment box is positioned in the cannulation robot (320g). The equipment box, in particular its seal, is automatically checked for soundness in order to guarantee the sterility of the contents (320h). The cannulation robot registers the completeness and sterility of the equipment, it is now ready for cannulation—this information is shown in a user display of the cannulation robot (320i). As a safety step, it is provided for a healthcare professional assisting the patient to be authenticated in order to brief the patient and authorize the cannulation robot for the patient (320j). Steps 320k to 320m are performed parallel to steps 320e to 320j. The system transmits patient data, particularly vascular structure data and planned venipuncture sites, in particular from the patient database, to the data processing device of the cannulation robot (320k), the consistency of the transmitted data is checked (320l), and the transmission either repeated until consistent or confirmed if transmitted accurately (320m).

The system checks the preparation status (320n) and informs personnel in the event of problems (320p), or (320o) decides to continue the treatment (320q).

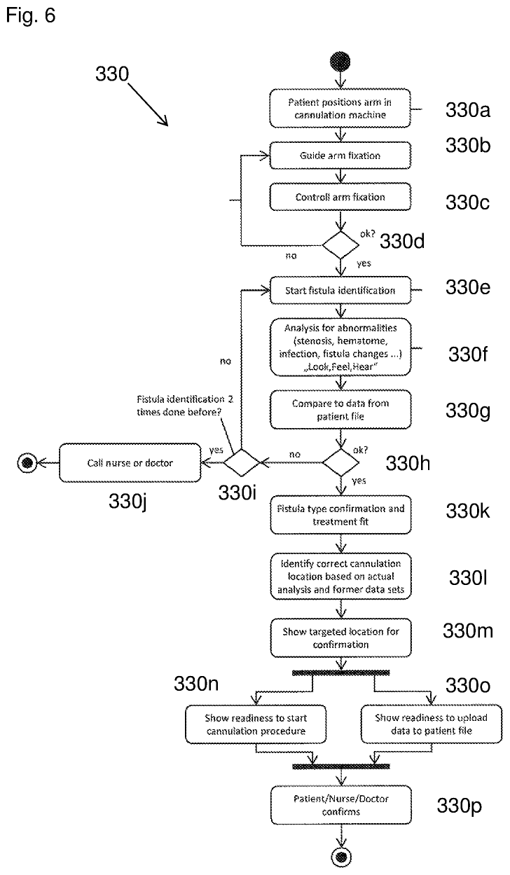

FIG. 6 shows an example embodiment of vascular identification step 330 (fistula identification):

The patient positions his arm in the treatment chamber of the cannulation robot (330a). The fixation device positions the fixing elements (330b) and checks the arm fixation (330c). If the event of insufficient fixation, a readjustment is made (330d). Identification of the fistula commences (330e). The vascular structure measuring device detects at least one image of the vascular structure containing the fistula, this being known on the basis of the patient data, and automatically checks the image for abnormalities, e.g. stenosis, hematoma, infections or pronounced fistula changes (330f). The identifying data is compared to historical identifying data from the patient data set (330g). Upon discrepancies, the automatic vascular identification is repeated and if again unsuccessful or abnormal (330i), a physician called in (330j). When the fistula is identified as per the historical data without abnormalities, the fistula and cannulation technique is confirmed (330k). The proper venipuncture site is then determined on the basis of the preceding preparatory analysis and/or by comparing the current vascular structure data to historical data (330l). The identified venipuncture site is shown on a display of the cannulation robot in relation to an image of the arm—in particular for confirmation by personnel for safety reasons (330m). The readiness of the automatic cannulation is displayed (330n) and, parallel thereto, the readiness to transmit patient data to the database. A physician or authenticated healthcare professional is prompted to confirm (330p).

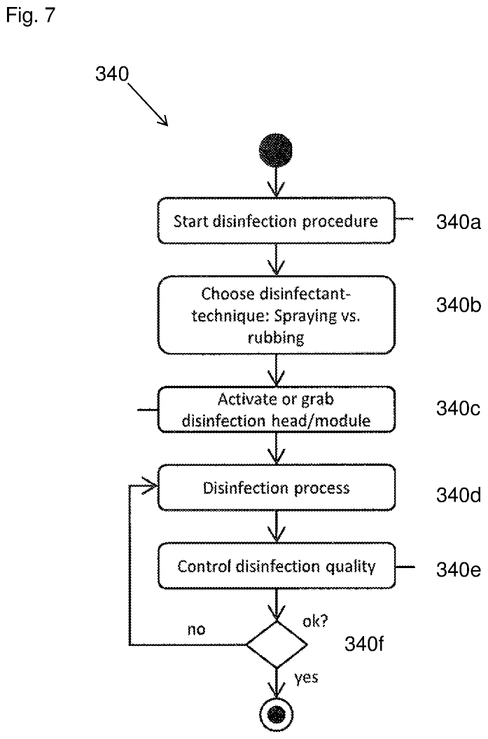

FIG. 7 shows an example embodiment of step 340 for the automatic disinfecting of the skin of the arm to be cannulated:

Automatic disinfection by the disinfecting device of the cannulation robot is started (340a). The appropriate disinfection technique is selected in patient and/or treatment-dependent manner (spraying or wiping with a swab; 340b). The disinfection tool head is activated or grasped by the tool arm (340c), the disinfection process begins (340d). The quality of the disinfection is checked by a testing device for disinfection control, an optional component of the cannulation robot and/or the disinfecting device (340d). If the check is positive, disinfection is terminated, otherwise the disinfection and test process is repeated (340f).

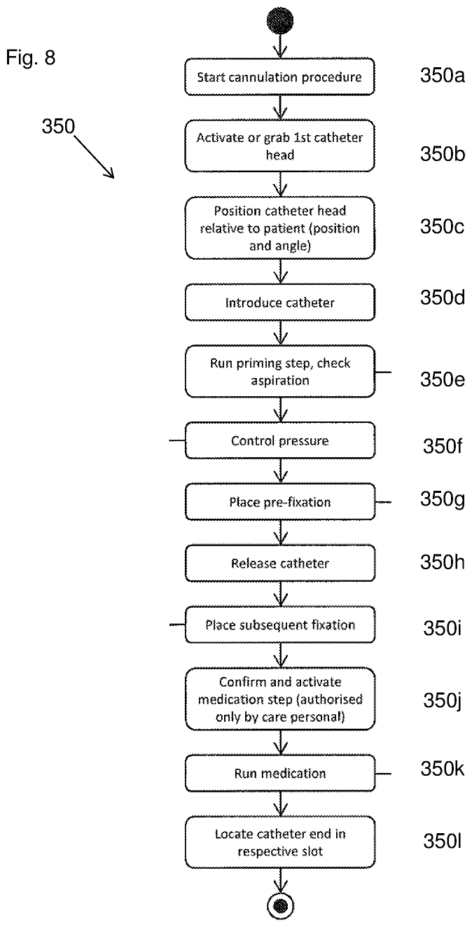

FIG. 8 shows an example embodiment of step 350 for the first venipuncture and cannulation of the skin of the arm to be cannulated:

The first automated cannulation begins (350a). The tool arm grabs the first cannula tool head equipped with the preselected patient and/or treatment-dependent cannula (350b). The cannula head is positioned relative to the arm 30 and to the predetermined puncture site, in particular the angle of the cannula is thereby aligned relative to the skin surface at the puncture site in a predetermined manner (350c). The cannula is inserted into the skin and the fistula (cannulation; 350d). A “priming step” is automatically performed to test the fluid connection between the cannula and the fistula by means of an aspiration test (350e). The pressure in the cannula is checked by means of a pressure measuring device of the cannulation robot (3500. A fixation tape is provisionally positioned by means of a fixation tape device in order to pro-visionally immobilize the cannula and/or catheter (350g). The cannula head releases the cannula (350h), the fixing of the cannula is subsequently reinforced by the fixation tape device (350i). Medication is planned subject to the authorization of authorized personnel (350j) and then automatically administered to the patient by a medicating device of the cannulation robot (350k). The end of the catheter is positioned in a seating in preparation of connecting to the hemodialysis machine (350l).

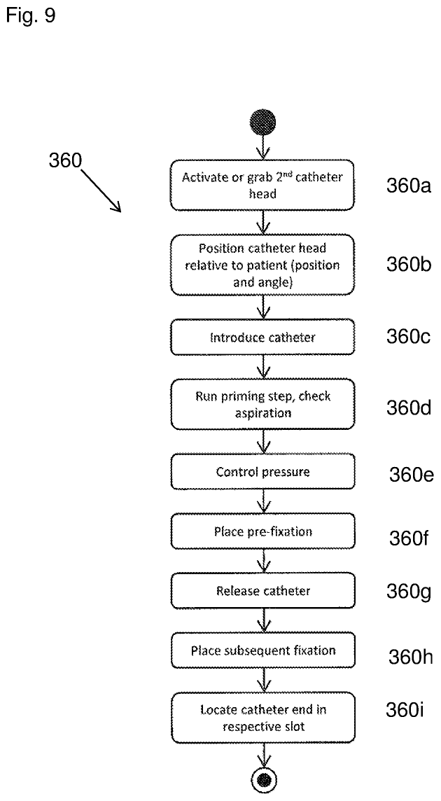

FIG. 9 shows an example embodiment of step 360 for the second venipuncture and cannulation of the skin of the arm to be cannulated:

The tool arm grabs the second cannula tool head equipped with the second preselected patient and/or treatment-dependent cannula (360a). The cannula head is positioned relative to the arm 30 and to the predetermined puncture site, in particular the angle of the cannula is thereby aligned relative to the skin surface at the puncture site in a predetermined manner (360b). The cannula is inserted into the skin and the fistula (cannulation; 360c). A “priming step” is automatically performed to test the fluid connection between the cannula and the fistula by means of an aspiration test (360d). The pressure in the cannula is checked by means of a pressure measuring device of the cannulation robot (360e). A fixation tape is provisionally positioned by means of a fixation tape device in order to provisionally immobilize the cannula and/or catheter (350f). The cannula head releases the cannula (360g), the fixing of the cannula is subsequently reinforced by the fixation tape device (360h). The end of the second catheter is positioned in another seating in preparation of connecting to the hemodialysis machine (360i).

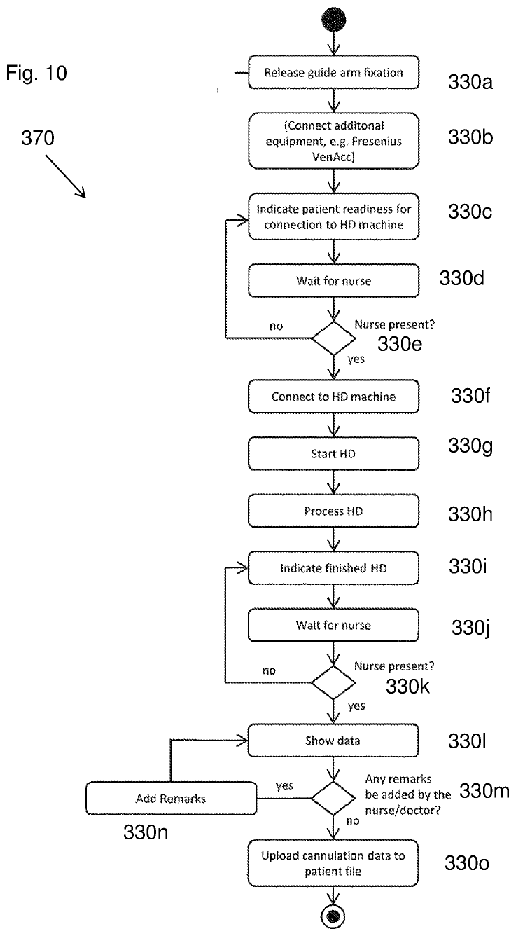

FIG. 10 shows an example embodiment of hemodialysis step 370, with substeps carried out by means of appropriately designed optional system apparatus:

The arm is released from the fixation of the cannulation robot (330a). Additional equipment is connected, e.g. Fresenius VenAcc (330b). The readiness of the patient to be connected to the hemodialysis machine is indicated in the display (330c). The readiness of the medical personnel is awaited (330d). If their readiness is not registered (330e), steps 330c and 330d are repeated. After registering the readiness of the medical personnel, the connection to the hemodialysis machine is made (330f), hemodialysis started (330g) and performed (330h). At the end of the hemodialysis, its completion is displayed (330i); medical personnel are awaited (330j). If their readiness is not registered (330k), steps 330i and 330j are repeated. After registering the readiness of the medical personnel, data on the hemodialysis is displayed (330m), which the personnel can supplement if needed (330n) and redisplay. The data is ultimately added to the patient data and stored in the database 330o).

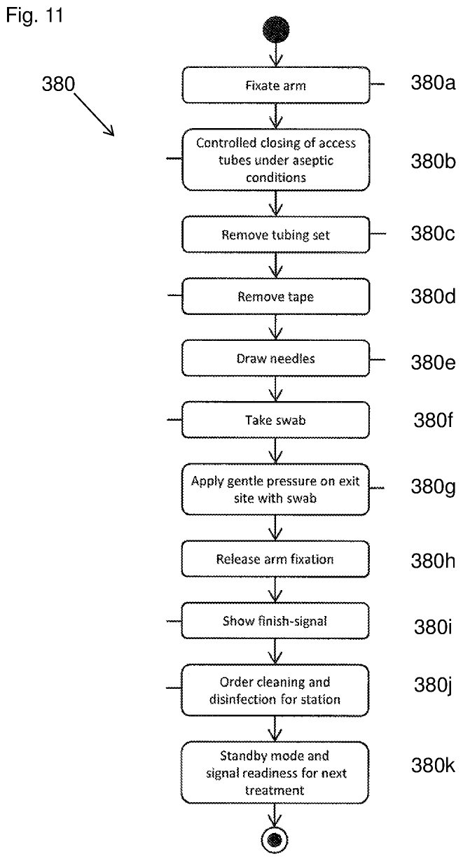

FIG. 11 shows an example embodiment of post-treatment step 380, with substeps carried out by means of appropriately designed optional system apparatus:

The arm of the patient is fixated (380a), the access tubes of the blood guiding system are closed in controlled manner under aseptic conditions (380b). The tubing set is removed (380c), the fixation tape is removed (380d), the cannulas are withdrawn and removed in controlled manner under aseptic conditions (380e). A swab is grasped (380f) and a gentle pressure applied to the puncture site with the swab (380g). The arm fixation is released (380h), a finish signal is displayed for the patient (380i). A cleaning and disinfection process is prompted to clean and disinfect the cannulation robot (380j). The cannulation robot is reset into a standby mode and its readiness for the next cannulation registered in the system and/or displayed (380k).

1. A system for data-dependent automated cannulation of patient blood vessels, in particular for hemodialysis, comprising:
at least one cannulation robot configured for automated cannulating of patient blood vessels,
a control system comprising at least one data processing device and which is configured to implement a control procedure which controls the at least one cannulation robot subject to program parameters,
at least one user interface device enabling user input by means of which a patient is registered in the control system, whereby in consequence of this control system registration procedure, an individually assigned patient identifier, which is referred to as the registered patient identifier, is used for the registered patient,
and wherein the control system is configured to define the program parameters as a function of the registered patient identifier and control the at least one cannulation robot as a function of the registered patient identifier.

2. The system according to claim 1, which comprises a patient database and a data storage apparatus, in which the patient database is stored, which contains the patient data sets of a plurality of patients, in each case comprising at least one patient identifier.

3. The system according to claim 1, wherein the at least one cannulation robot is configured to automatically perform a first venipuncture for insertion of a first cannula into a blood vessel for automatic withdrawing and routing of the blood to a blood guiding system.

4. The system according to claim 3, wherein the at least one cannulation robot is configured to automatically perform a second venipuncture for insertion of a second cannula into a blood vessel for automatically returning the blood from the blood guiding system for performing hemodialysis.

5. The system according to claim 1, wherein the control system is configured to implement a selection process for selecting the program parameters to be used for the automated cannulation as a function of the registered patient identifier.

6. The system according to claim 1, wherein the selection process is configured to access a data matrix in which the necessary program parameters are linked based on treatment data and thereby determinable, wherein the system comprises a data storage apparatus in which the data matrix is stored.

7. The system according to claim 1, wherein the control system is configured to implement at least one preparatory procedural step after the recording of the registered patient identifier and prior to the start of the automated cannulation as a function of the registered patient identifier which prepares the automatic withdrawal of the blood sample from the patient's blood vessel by means of the cannulation.

8. The system according to claim 1, wherein the control system is configured to implement at least one accompanying procedural step during the withdrawing of the blood sample obtained by the automated cannulation as a function of the registered patient identifier which is performed at least in part or entirely parallel to the cannulation.

9. The system according to claim 1, wherein the control system is configured to implement at least one secondary procedural step after the start of the automated cannulation and/or after the completion of the cannulation of the at least one blood vessel and/or after withdrawal of the blood sample obtained by means of the automated cannulation as a function of the registered patient identifier.

10. The system according to claim 1, wherein the system comprises an identification system which detects the position and/or dimensions and/or condition of the body part to be venipunctured positioned in the treatment chamber, continuously and/or in real time, by means of a sensor system based on measuring radiation and/or light and/or ultrasound, and stores it in the form of identifying data.

11. The system according to claim 1, wherein the system comprises an identification device for detecting identifying data providing information on the position, dimensions and/or condition of the body part to be cannulated and/or the blood vessel of the patient and a data storage apparatus for storing the identifying data as a function of the registered patient identifier, wherein the system is configured to prepare a further automated treatment by applying historical data containing the stored identifying data as a function of said registered patient identifier.

12. The system according to claim 1, wherein the at least one user interface device is positioned or positionable at a different geographic location than the at least one cannulation robot.

13. The system according to claim 1 comprising a data storage apparatus which is positioned at a different geographic location than the at least one cannulation robot and the at least one user interface device.

14. The system according to claim 1, wherein the control system is designed to register the registering patient as a function of successful system authentication, ascertain a patient identifier and/or recognize patients as registered patients by identifying the registered patient identifier.

15. A method for data-dependent automated cannulation of patient blood vessels comprising the following steps:
Recording a registered patient identifier upon a patient registering at a user interface device which is data-linked to a data processing control system;
Determining program parameters as a function of the registered patient identifier by the control system; and
Controlling a cannulation robot set up for the automated cannulation of patient blood vessels and which is data-linked to the control system as a function of the program parameters for the automated cannulation of a patient's blood vessel based on the patient's registered patient identifier.
